(19) United States
(12) Patent Application Publication
(10) Pub. No.: US 2014/0086961 A1
(43) Pub. Date: Mar. 27, 2014

(54) USE OF CYCLODEXTRINS IN DIETS, WATER OR VACCINE ADJUVANTS TO BOOST THE IMMUNE SYSTEM OF FISH

(71) Applicants: Frederick W. Goetz, Shorewood, WI (US); Rebecca Klaper, Glendale, WI (US); Simon MacKenzie, Figaro-montmany (ES)

(72) Inventors: Frederick W. Goetz, Shorewood, WI (US); Rebecca Klaper, Glendale, WI (US); Simon MacKenzie, Figaro-montmany (ES)

(73) Assignees: UNIVERSITAT AUTONOMA DE BARCELONA, Barcelona (ES); UWM RESEARCH FOUNDATION, INC., Milwaukee, WI (US)

(21) Appl. No.: 14/116,460

(22) PCT Filed: May 8, 2012
(86) PCT No.: PCT/US2012/036938

§ 371 Date: Nov. 8, 2013

Related U.S. Application Data
(60) Provisional application No. 61/483,895, filed on May 9, 2011.

(51) Int. Cl. A61K 31/724; A23K 1/16; A23K 1/18

(52) U.S. Cl. CPC A61K 31/724; A23K 1/188; A23K 1/1643
USPC 424/278.1

(57) ABSTRACT

The present disclosure describes compositions containing cyclodextrin and methods of using cyclodextrins to stimulate or enhance the immune system and response in fish. Methods of enhancing the efficacy of a fish vaccine by administering cyclodextrin to the fish are also described.

### CROSS-REFERENCE TO RELATED APPLICATIONS

This application claims priority to U.S. Provisional Patent Application Ser. No. 61/483,895, filed May 9, 2011, and which is incorporated by reference herein in its entirety.

### STATEMENT REGARDING FEDERALLY SPONSORED RESEARCH

This invention was made with government support under grant number RD833319 awarded by the United States Environmental Protection Agency and grant number 58-3655-9-748 awarded by United States Department of Agriculture-Agricultural Research Service. The United States government has certain rights in the invention.

### INTRODUCTION

There are two major parts to the immune system in vertebrates: the Innate and the Adaptive. The innate immune system is the first to react to a pathogen intrusion to the body, and while the reaction is far less specific than the antibody response of the adaptive immune system, it can be a powerful and a quick block to infection. The innate immune system reacts to different classes of pathogens (e.g., Gram-negative bacteria versus viruses) in different ways. There has been significant research on methods that can be used to “boost” the innate immune system of vertebrates. The idea in this case is to charge the innate immune system in the event of an impending infection or to be able to address an infection more intensely. For humans, there has been a great deal of research on the ability of glucans that are present in yeast and other products to be used for this purpose. They have been shown experimentally to increase the activity of the innate immune system and have been demonstrated clinically to protect against infection. This same concept has been adopted in the aquaculture Industry. There have been fish diets produced (e.g., BioMar's “EcoActiva” and EWOS' “EWOS Boost” (yeast B-glucans)) that have agents in them that are reported to boost the immune system and a number of studies have also been conducted to look at dietary glucan or other additives.

Few compounds are available for disease treatment and prevention in aquatic organisms. There is a lack of vaccines and well worked out prophylaxis regimens for pet fish. Currently, only four compounds are approved by the FDA for use in aquatic species and the use is extremely limited in terms of species, indication, and route of administration. The present technologies for either treating or protecting fish from bacteria are antibiotics that act against the pathogen itself or the use of prophylactic agents such as glucans that supposedly activate the immune system. Antibiotics work on the pathogen and not the immune system of the host. The overuse of antibiotics has the public looking for safer alternatives to disease treatment. Once fish disease is recognized it is often too late for effective treatment. There is a lack of data showing efficacy for commercially available fish immune boosting products.

### SUMMARY

In one embodiment, a method of stimulating the innate immune system of fish is disclosed. The method includes feeding fish a composition comprising an amount of cyclodextrin effective to stimulate the innate immune system of the fish with the proviso that the composition does not comprise cysteamine.

In another embodiment, a method of stimulating the innate immune system of fish includes combining the fish with water comprising cyclodextrin at a concentration effective to stimulate the innate immune system of the fish.

In another embodiment, a method to enhance the efficacy of a fish vaccine includes administering a cyclodextrin to a fish treated with the vaccine.

In another embodiment, the use of cyclodextrin to stimulate the innate immune system of the fish is disclosed.

In another embodiment, the use of cyclodextrin to enhance the efficacy of a fish vaccine is disclosed.

Other aspects of the invention will become apparent by consideration of the detailed description and accompanying drawings.

### BRIEF DESCRIPTION OF THE DRAWINGS

FIG. 1 shows the effects of beta cyclodextrin (CD) attached to C60 fullerene, and beta cyclodextrin by itself on IL-1B gene expression in trout macrophages. LPS is a positive stimulatory control. Macrophages were isolated from head kidneys of rainbow trout, plated and then stimulated with the preparations for 24 hours. RNA was extracted from cells and analyzed by QPCR for IL-1B expression. Data are means for 3 independent trials.

FIG. 2 shows the effects of beta cyclodextrin attached to C60 fullerene (C60/BD) and beta cyclodextrin alone (CD) on cell viability measured by the ability of metabolically active cells to reduce resazurin to resorufin, a highly fluorescent product. High fluorescence indicates no loss in cell viability. Cells were the same ones used in FIG. 1. Saponin is a positive control since it kills cells.

FIG. 3 shows reverse transcription (RT-PCR) assay results from an experiment in which trout macrophages were stimulated with LPS, beta cyclodextrin (CyD), beta cyclodextrin and LPS (CyD/LPS), no agents (C=control) or no DNA template (C−=no template control). Cells were stimulated for 24 hours and RNA extracted and RT-PCR conducted with TNF (tissue necrosis factor), Mx protein, IL-6 (interleukin 6), IL-1B (interleukin 1B), TLR (toll-like receptor), IFN (interferon), CD18 (cluster of differentiation 18), and PU1 (transcription factor PU1). 18S is a RNA loading control. Molecular weight standard in first lane.

FIG. 4 shows the effects of different cyclodextrins on IL-1B and IL-6 gene expression in rainbow trout macrophages. Trout macrophages were stimulated for 30 minutes to 24 hours with heptakis (2,6-di-O-methyl)-β-cyclodextrin (Heptakis); methyl-β-cyclodextrin (Methyl β); beta cyclodextrin (β CyD); 2-hydroxypropyl-β-cyclodextrin (Hydroxy β); 2-hydroxypropyl-α-cyclodextrin (Hydroxy α); gamma cyclodextrin (γ CyD). RNA was extracted and RT-PCR conducted with IL-1B and IL-6. 18s was a RNA loading control. Molecular weight standard in first lane

FIGS. 5A and 5B show replicates of the effects of α-cyclodextrin at various concentrations on LPS challenge. Zebrafish larvae, 2 days post-fertilization, were bathed with 0, 67.5, 125, 250 or 500 μg/mL of alpha cyclodextrin. Zebrafish larvae were then bathed with a lethal concentration (100% mortality) of LPS (150 μg/mL—Pseudomonas aeruginosa) and assessed for mortality over 36 hours. FIGS. 5A and 5B show the cumulative mortality after challenge of zebrafish larvae treated with α-cyclodextrin

FIGS. 6A and 6B show replicates of the effects of β-cyclodextrin at various concentrations on LPS challenge performed in replicate. Zebrafish larvae, 2 days post-fertilization, were bathed with 0, 67.5, 125, 250 or 500 μg/mL of beta cyclodextrin. Zebrafish larvae were then bathed with a lethal concentration (100% mortality) of LPS (150 μg/mL—Pseudomonas aeruginosa) and assessed for mortality over 36 hours. FIGS. 6A and 6B show the cumulative mortality after challenge of zebrafish larvae treated with β-cyclodextrin

FIG. 7 shows survival toxicity data of cyclodextrin treatment post to first feeding.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The present disclosure generally relates to compositions containing one or more cyclodextrins that may be fed, supplied or administered to fish, as well as methods of treating fish or stimulating fish immunity by administering one or more cyclodextrins. The present inventors surprisingly discovered that cyclodextrins are potent inducers of pro-inflammatory gene expression in fish. The compositions described herein may be used as a prophylactic or therapeutic treatment for diseases of fish. The compositions may be used to protect fish against infections particularly when the fish are exposed to stressful situations that can result in increased susceptibility to disease. For example, the use of a composition containing cyclodextrin in a fish macrophage model system stimulates significant increases in several inflammatory cytokines which aid in fighting infection. Zebrafish larvae treated with cyclodextrin-containing water and challenged with a lethal concentration of bacterial lipopolysaccharide (LPS) are able to survive the insult.

Cyclodextrin is a cyclic oligomer of alpha-D-glucopyranose. As used herein, “alpha cyclodextrin” and “α-cyclodextrin” refer to a six-member sugar ring molecule; “beta cyclodextrin” and “β-cyclodextrin” refer to a seven-member sugar ring molecule; and “gamma cyclodextrin” and “γ-cyclodextrin” refer to an eight-member sugar ring molecule. In some embodiments, “cyclodextrin” includes cyclodextrin and/or its derivatives, including, but not limited to, alkyl and hydroxyalkyl derivatives. Examples of cyclodextrins include, but not limited to, alpha cyclodextrin, beta cyclodextrin, gamma cyclodextrin, methyl β-cyclodextrin (also referred to as “Methyl β”), random methyl β-cyclodextrin, hydroxypropyl-β-cyclodextrin (also referred to as “Hydroxy β” and “2-hydroxypropyl-β-cyclodextrin”), hydroxyethyl β-cyclodextrin, polycyclodextrin, heptakis(2,6-di-O-methyl)-β-cyclodextrin (also referred to as “Heptakis”), Heptakis(2,3,6-tri-O-Methyl)-β-Cyclodextrin, Heptakis(6-Amino-6-Deoxy)-β-Cyclodextrin, Heptakis(2,3,6-tri-O-Benzoyl)-β-Cyclodextrin, hydroxyethyl β-cyclodextrin, 2-hydroxypropyl-α-cyclodextrin (also referred to as “Hydroxy a”), hydroxypropyl-γ-cyclodextrin, hydroxyethyl-γ-cyclodextrin, random methyl-γ-Cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-β-cyclodextrin, maltotriosyl-β-cyclodextrin, maltotriosyl-γ-cyclodextrin, dimaltosyl-β-cyclodextrin, mono- or polyalkylated β-cyclodextrin, mono- or polyhydroxyalkylated β-cyclodextrin, mono, tetra or hepta-substituted β-cyclodextrin, succinyl-cyclodextrins, including Succinyl α-Cyclodextrin, Succinyl β-Cyclodextrin, and Succinyl γ-Cyclodextrin, succinyl-(2-hydroxypropyl)-cyclodextrins, including Succinyl-(2-Hydroxypropyl)-α-Cyclodextrin and Succinyl-(2-Hydroxypropyl)-β-Cyclodextrin, carboxymethylcyclodextrins, including Carboxymethyl β-Cyclodextrin, sulfobutylcyclodextrins, including Sulfobutyl β-cyclodextrin, aminocyclodextrin, dimethylcyclodextrin, cyclodextrin phosphates, or salts thereof, including α-Cyclodextrin Phosphate, β-Cyclodextrin Phosphate, γ-Cyclodextrin Phosphate, hydroxyethylcyclodextrin, acetyl-cyclodextrin, including Acetyl β-Cyclodextrin, ethylcyclodextrins, trimethylcyclodextrins, carboxyethylcyclodextrin, glucosylcyclodextrin, 6-O-α-maltosylcyclodextrins, butyl-cyclodextrins, sulfated cyclodextrins, or salts thereof, including α-Cyclodextrin Sulfate, β-Cyclodextrin Sulfate and γ-Cyclodextrin Sulfate, N,N-diethylaminoethylcyclodextrin, tert-butylsilylcyclodextrins, Silyl[(6-O-tert-butyldimethyl)-2,3,-di-O-acetyl)-cyclodextrins, Sulfopropyl-cyclodextrins, 6-Monodeoxy-6-Monoamino-β-Cyclodextrin Hydrochloride, polycyclodextrins, sulfoalkyl ether cyclodextrin, soluble α-cyclodextrin polymer crosslinked with epichlorohydrin, soluble β-cyclodextrin Polymer crosslinked with epichlorohydrin, soluble γ-cyclodextrin polymer crosslinked with epichlorohydrin, soluble anionic β-cyclodextrin polymer crosslinked with epichlorohydrin and substituted by carboxymethyl groups and branched cyclodextrin.

The term “effective amount,” as used herein, refers to the amount of cyclodextrin necessary to elicit the desired biological response. In accordance with the subject invention, the effective amount of cyclodextrin is the amount necessary to reduce or prevent the incidence of disease in fish. In some embodiments, the effective amount of cyclodextrin is the amount necessary to treat or ameliorate a disease in fish. For example, the effectiveness of the cyclodextrin may be determined by monitoring or measuring a change in a particular characteristic and/or diagnosing a symptom of the particular disease. For example, the expression levels of genes involved in the innate immune system may be monitored or measured to determine if there is an increase or decrease in the expression levels. A decrease in expression levels may indicate an activation of the innate immune system, if the decrease in expression levels occurs in a gene that inhibits the innate immune system. A decrease of at least 10%, at least 20%, at least 30%, at least 40%, at least 50%, at least 60%, at least 70%, at least 80%, at least 90%, at least 95%, at least 98% or at least 100% in expression levels of one or more innate immune system genes may indicate activation of the innate immune system. An increase in expression levels may indicate an activation of the innate immune system. An increase of at least 10%, at least 20%, at least 30%, at least 40%, at least 50%, at least 60%, at least 70%, at least 80%, at least 90%, at least 95%, at least 98% or at least 100% may indicate activation of the innate immune system.

In some embodiments, the protein or transcript expression levels of genes involved in the innate immune system may be monitored or measured. Examples of genes involved in the innate immune system include but not limited to Mx, Stat1a, Stat1b, Gig2, NF-kappa-B, transforming growth factors (TGFs), interferon regulatory factors (IRFs), interferons (IFNs), interleukin 6 (IL-6), tissue necrosis factor alpha (TNFa), and interleukin 1 B (I-1B). In particular embodiments, the genes may be selected from genes involved in the pro-inflammatory responses, such as IL-6, TNFs, and IL-1B. In particular embodiments, the genes may be selected from genes involved in viral responses, such as IFNs and Mx. In some embodiments, the expression level may be measure in tissues, such as skin, gills, intestine or spleen. The gene expression may be monitored by a microarray containing many immune- or stress-related genes.

As used herein, a “stress event” describes events that may cause stress to the fish and result in impaired immune function and hence result in infection. Examples of stress events include, but not limited to, sorting, grading, moving of fish and water changes.

In an aspect, the disclosure describes a method of stimulating the innate immune system of fish. In one embodiment, the method includes feeding the fish a composition including a component that is digestible or non-toxic to fish and an effective amount of cyclodextrin to stimulate the innate immune system of the fish. The composition typically does not include any other active agent, such as cysteamine, i.e., the composition is substantially free of an active agent such as cysteamine. As used herein, “cysteamine” includes cysteamine, cysteamine salts (such as cysteamine hydrochloride and cysteamine phosphate), as well as analogs, derivatives, conjugates, and metabolites of cysteamine. As used herein, an “active agent” is a pharmacologically active substance other than cyclodextrin that produces a localized or systemic effect in fish. Examples of active agents include antibiotics, anti-fungals and anti-viral agents. In one embodiment, the method includes administering to the fish a composition including cyclodextrin in an effective amount to stimulate the innate immune system of the fish, wherein the composition is added to water in which the fish is immersed. As used herein, “substantially free” means that the amount of active agent present in the composition is zero or is lower than that needed to have a pharmacological effect when administered to fish.

In one aspect, described are methods of reducing or preventing the incidence of developing, or treating or ameliorating disease in fish. The method can include feeding the fish a composition containing an effective amount of cyclodextrin to reduce, prevent, treat or ameliorate the disease in fish. The composition may be added to fish food, or may include fish food. In one embodiment, the method includes administering cyclodextrin to fish by adding cyclodextrins directly to the water in which the fish are immersed. The amount of cyclodextrin added to the water is effective to reduce, prevent, treat or ameliorate the disease in fish.

Disclosed are methods in which a composition containing cyclodextrin is fed to the fish prior to the stress event, as well as methods in which the composition containing cyclodextrin is fed to the fish at the onset of the symptoms of disease. In some embodiments, cyclodextrins may be added to diets that are fed to commercially aquacultured fish stocks prior to times when fish could be stressed due to grading, sorting, transport or handling and are, therefore, more susceptible to infection.

In some embodiments, the compositions contain fish food, which can include plant or animal material intended for consumption by fish, or a combination thereof. The fish food can contain macro nutrients, trace elements, and vitamins necessary to keep the fish in good health. The fish food can also contain additives. The fish food can be in flake, pellet or tablet form.

In some embodiments, cyclodextrins either alone, or in a composition comprising one or more additional components, may be added to the water in contact with the fish for treating fish prior to a stress event and as a general prophylactic against infection. In some embodiments, the composition containing cyclodextrins may be added to the water to attain a certain level of composition in the water such that the fish is continuously treated with the composition. The concentration of cyclodextrin may be at least about 5, at least about 10, at least about 15, at least about 20, at least about 25, at least about 30, at least about 40, at least about 45, at least about 50, at least about 100, at least about 150, at least about 200, at least about 250, at least about 300, at least about 400, or at least about 500 μg/mL (w/v), and less than about 1500, less than about 1200, less than about 1000, less than about 750, less than about 600, less than about 500, less than about 400, or less than about 300 μg/ml (w/v). In some embodiments, the composition containing cyclodextrins may be administered at the onset of the symptoms of disease.

Dosage regimens of cyclodextrin may be adjusted to provide the optimum desired response (e.g., a therapeutic or prophylactic response). A suitable dosage range may, for instance, be about 5 mg/kg to about 500 mg/kg, about 5 mg/kg to about 50 mg/kg, about 25 mg/kg to about 75 mg/kg, about 50 mg/kg to about 100 mg/kg, about 75 mg/kg to about 125 mg/kg, about 100 mg/kg to about 150 mg/kg, about 125 mg/kg to about 175 mg/kg, about 150 mg/kg to about 200 mg/kg, about 175 mg/kg to about 225 mg/kg, about 200 mg/kg to about 250 mg/kg, about 225 mg/kg to about 275 mg/kg, about 250 mg/kg to about 300 mg/kg, about 275 mg/kg to about 325 mg/kg, about 300 mg/kg to about 350 mg/kg, about 325 mg/kg to about 375 mg/kg, about 350 mg/kg to about 400 mg/kg, about 375 mg/kg to about 425 mg/kg, about 400 mg/kg to about 450 mg/kg, about 425 mg/kg to about 475 mg/kg, or about 450 mg/kg to about 500 mg/kg body weight.

In some embodiments, fish behaviour may be monitored and recorded to determine the effectiveness of the composition containing cyclodextrin. Examples of behaviour include swimming activity, tank distribution and response to stress stimuli, e.g., escape response to netter. In some embodiments, a pathological exam may be performed to identify lesions, intestinal rigidity, integrity and status of internal organs.

The fish can be marine or salt-water fish. The fish can be tropical fish. Examples of suitable fish for the method of invention include salmonids (Oncorhynchus sp., including rainbow trout, and Salmo sp., including Atlantic salmon), American, European, and Japanese eels (Anguilla sp.), tilapia (Oreochromis sp.), striped bass and hybrid-striped bass (Morone chrysops. and M. saxatilis), flounders (Seriola sp. including Citharidae, Scophthalmidae (turbots), Bothidae (lefteye flounders), Pleuronectidae (righteye flounders), Paralichthyidae (large-tooth flounders), Achiropsettidae (southern flounders), Samaridae, Soleidae (true soles), and Achiridae (American soles)), seabream (Sparus sp.), sea perch (Lates calcarifer), the estuarine grouper (Epinephelus tawine), walleye (Stitzostedion vitreum), yellow perch (Perca flavescens), channel catfish (Ictalurus punctutus), centrachids (such as largemouth bass, Micropterus salmoides), brown bullheads (Nebulosus sp.), fat head minnows (Pimephales promelas), golden shiners (Netemigonus crysoleucas), goldfish (Carassius auratus), carp (Cyprinus carpio), and aquarium fish species such as zebrafish (Danio rerio), black mollies (Poecilia sphenops) and platies (Xiphosphorus maculatus).

In one aspect, the present invention is directed to the immuno stimulating and vaccine enhancing effects of cyclodextrins. Disclosed are methods to stimulate the efficacy of a fish vaccine that comprises administering a cyclodextrin to a fish treated with the vaccine. In one aspect the cyclodextrin may be included as an adjuvant in a vaccine for immunizing fish against disease. The purpose of the cyclodextrin adjuvant is to heighten the immune response of the fish to increase the effect of the vaccination and the subsequent stimulation of the adaptive immune system and production of memory cells. The present disclosure also describes administering cyclodextrin to fish as a component of the adjuvant in the vaccine to increase the immune response during vaccination.

In some embodiments, the vaccine used to immunize the fish maybe a killed vaccine, an inactivated vaccine, an attenuated vaccine, a toxoid vaccine, a subunit vaccine, a conjugated vaccine and a DNA vaccine. In some embodiments, the vaccine and composition containing cyclodextrin may be delivered by intraperitoneal injection, by immersion or by oral administration. The vaccine may be effective against a bacterium, a virus or parasite.

Fish diseases caused by bacteria, viruses or parasites include, but not limited to, Lymphocystis Disease, Herpesvirus salmonis (Herpesvirus disease of Salmonids), Channel Catfish Virus, Epithelioma papillosum (Fish Pox), Infectious Hematopoietic Necrosis (IHN), Viral Hemorrhagic septicemia, Spring Viremia of Carp (SVC) and Swim Bladder Infection virus (SBI), pancreas disease (PD), sudden death syndrome (chronic PD), Infectious Pancreatic Necrosis (IPN), Bacterial Hemorrhagic Septicemia, Edwardsiella septicemia, Enteric septicemia of catfish, Furunculosis, Ulcerative disease of goldfish, Enteric red mouth, Columnaris disease or Saddleback disease, Bacterial Gill Disease, Rainbow Trout Fry Anemia, infectious salmon anemia, Bacterial Kidney Disease, Amylodinium (marine velvet), Anchor worms, Cryptocaryon (marine ick), Dactylogyrus (gill flukes), Dropsy, Fin rot, Gyrodactylus (skin flukes), lchthyophthirius (white spot or ick), Velvet Disease, Oodinium, Hexamita (hole in the head), Tuberculosis, heart and skeletal muscle inflammation, and Chlamydial infection.

In some embodiments, the vaccine is for a virus-based disease. Viruses which cause disease in fish include, but not limited to, Iridovirus, infectious spleen and kidney necrosis virus, herpesvirus, Channel Catfish Virus, Herpesvirus cyprinid, alphavirus (such as Salmon Pancreas Disease Virus (SPDV)), infectious salmon anemia virus (ISAV), piscine reovirus (PRV), Rhabdovirus, and Birnavirus (such as Infectious Pancreatic Necrosis Virus). In some embodiments, the vaccine is for a bacteria-based disease, such as a disease caused by a gram-negative bacteria. Bacteria which cause disease in fish include, but not limited to, Aeromonas hydrophila, Aeromonas salmonicida Pseudomonas fluorescens, Pseudomonas anquilliseptica, Pseudomonas aeruginosa, Vibrio sp., e.g., Vibrio septicemia, V. alginolyticus, V. anquillarum (also known as Listonella anguillarum), V. salmonicida, and V. damsela, Edwardsiella tarda, Edwardsiella ictaluri Aeromonas salmonicida, Yersinia ruckeri, Streptococcus iniae, Flexibacter columnaris, Flexibacter maritimus, Flexibacter psychrophilus, Flexibacter columnaris, Flavobacterium sp., Cytophaga psychrophila Renibacterium salmoninarum, Mycobacterium sp., Nocardia sp., and Epitheliocystis.

The examples, which are intended to be purely exemplary of the invention, and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (for example, amounts and temperature), but some experimental errors and deviations should be accounted for. Unless indicated otherwise, parts are parts by weight, molecular weight is average molecular weight, temperature is in degrees Centigrade, and pressure is at or near atmospheric. Examples and references are given below to illustrate the present invention in further detail, but the scope of the present invention is not limited by these examples. Any variations in the exemplified articles which occur to the skilled artisan are intended to fall within the scope of the present invention. Further examples of such combinations can be found throughout the specification, but would also be known to those of ordinary skill in the art in light of the present disclosure.

### EXAMPLES

### Example 1

RNA isolation Total RNA was extracted from individual zebrafish brains using 0.3 mL of TriReagent (Molecular Research Center) following manufacturer's instructions. RNA concentration was quantified (Nanodrop ND-1000) and RNA integrity and quality assessed (Bioanalyzer 2100, Agilent Technologies). The RNA integrity number (RIN) was calculated for each sample and only RNAs with a RIN number greater than 7 were processed. RNA (1 μg) was used to synthesize cDNA with SuperScript® III Transcriptase (Invitrogen) and oligo-dT primer (Promega).

Real-Time Quantitative PCR

Standard SYBR-green based methodology was used for anti-viral and pro-inflammatory gene expression in mucosal immune system (intestine/gills/skin/spleen) to evaluate effective dose and identify the effects of β-cyclodextrins on tissue activation profiles. Briefly, 2 μg of total RNA was used for cDNA synthesis (SuperScript® III, Invitrogen) and subsequently diluted with nuclease-free water to 1 ng/μL cDNA. Gene-specific high-melting temperature primers for genes of interest were designed using NCBI/Primer-BLAST suite. PCR reactions were conducted on an ABI 7900 Sequence Detection System (Applied Biosystems) using a hot start SYBR-green based method (Fast SYBR® Green Master Mix, ABI) followed by melting curve analysis to verify specificity of the product. All transcripts were normalized to the housekeeping gene 18s. Quantitative expression data between different times were examined by one-way ANOVA using SPSS 17 statistical software and the differences were considered significant at p<0.05.

### Example 2

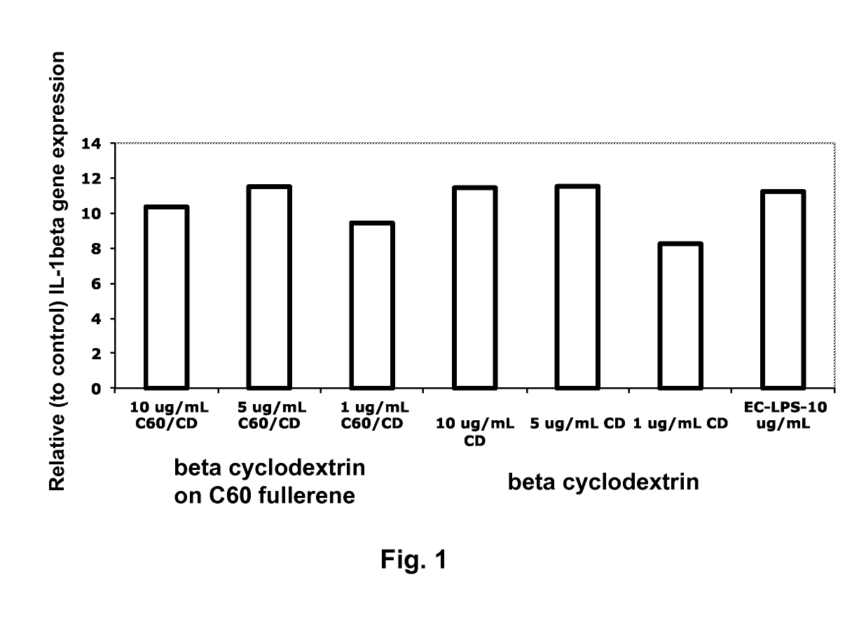

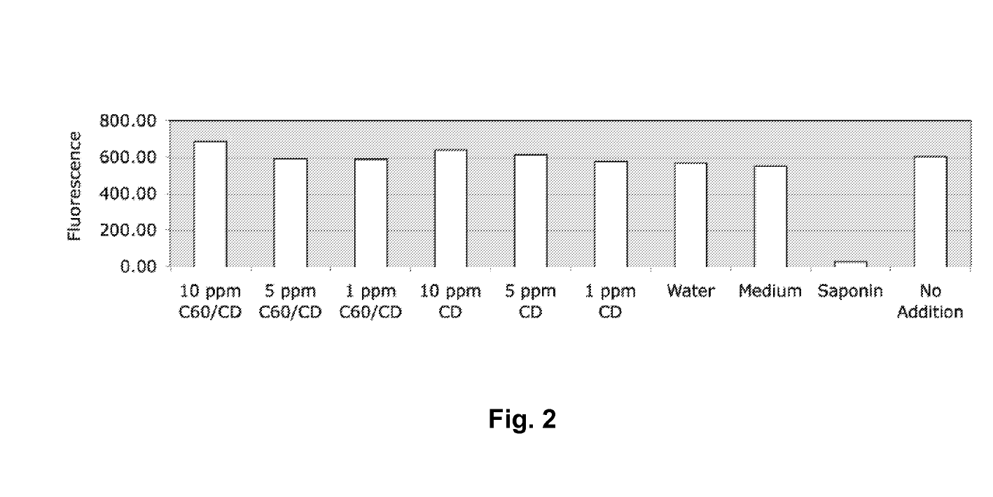

A primary trout macrophage culture system was used to investigate how pathogens are recognized by the innate immune system in fish (MacKenzie et al., Developmental and Comparative Immunology 27:393-400 (2003); Iliev et al., Molecular Immunology 42:1215-1223 (2005); Iliev et al. FEBS Letters 579 (29):6519-6528 (2005)). In this system, macrophages can be stimulated by various compounds and the expression of immune genes such as interleukin 1 B (IL-1B) or tissue necrosis factor (TNF) can be assayed as a measure of the degree of inflammatory stimulation and, thus, activation of the innate immune system. The effect of nanoparticles on the immune system of trout as an indication of the possible harmful effects of these compounds on aquatic organisms was studied. This involved exposing the cultured trout macrophages to nanoparticles and looking for effects on IL-1B and interferon alpha (IFNa) gene expression. Bacterial lipopolysaccharide (LPS) was used as a positive stimulatory control. Macrophages were isolated from head kidneys of rainbow rout, plated and then stimulated with the preparations for 24 hours. RNA was extracted from cells and analyzed by QPCR for IL-1B expression. Data are means+/−SD for 3 independent trials. In the process, one compound, a fullerene (C60) that had beta cyclodextrin attached to it, was a potent inducer of IL-1B expression (FIG. 1), similar to the bacterial LPS positive control. However, when the beta cyclodextrin was applied by itself to the cells in the same quantity, it surprisingly produced the same effect as the fullerene/beta cyclodextrin combination (FIG. 2). While fullerenes do have an effect on IL-1B expression by themselves, it was clear from these results that beta cyclodextrin by itself was also a potent stimulator of inflammatory gene expression in trout macrophages. The effect of beta cyclodextrin on any cell viability was determined using the QBlue Cell Viability Assay Kit (BioChain) and saponin as a positive control for cell death. No effect on viability as measured by this assay was seen (FIG. 2), indicating that the application of either cyclodextrin attached to C60 or cyclodextrin alone did not lead to a loss in cell viability.

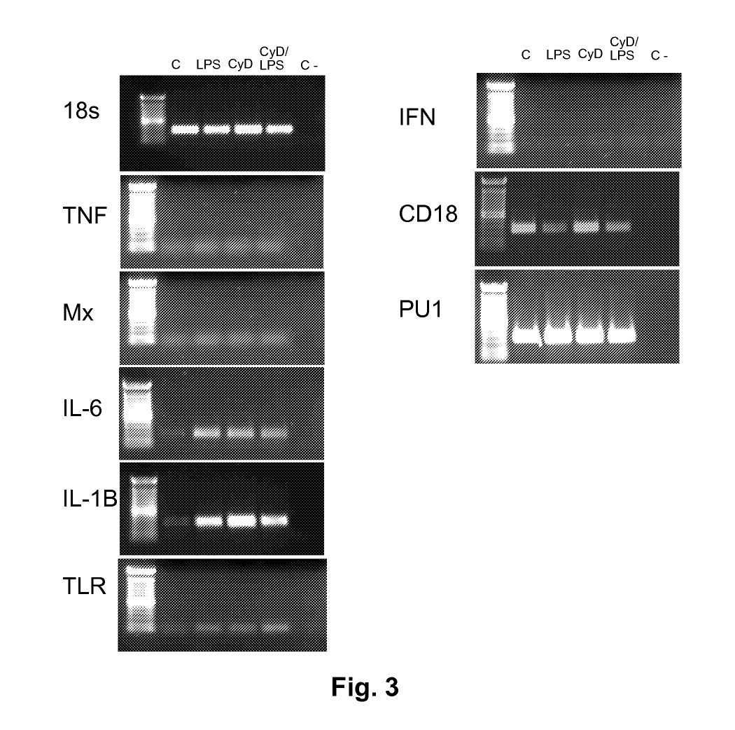

The effects of cyclodextrin on the expression of several other genes such as TNF and IL-6 that are also pro-inflammatory were studied (FIG. 3). FIG. 3 shows RT-PCR assay results from an experiment in which trout macrophages were stimulated with LPS, beta cyclodextrin (CyD), beta cyclodextrin and LPS (CyD/LPS), no agents (C=control) or no DNA template (C-=no template control). Cells were stimulated for 24 hours and RNA extracted and RT-PCR conducted with TNF (tissue necrosis factor), Mx protein, IL-6 (interleukin 6), IL-1B (interleukin 1B), TLR (toll-like receptor), IFN (interferon), CD18 (cluster of differentiation 18), and PU1 (transcription factor PU1). 18S is a RNA loading control. Molecular weight standard is in the first lane.

For beta cyclodextrin, IL-1B was the most highly stimulated gene. Interestingly, while beta cyclodextrin stimulates inflammatory gene expression in trout macrophages (see Motoyama et al., FEBS Letters 579:1707-1714 (2005)), in mammals, cyclodextrins appear to have an opposite effect. Cyclodextrins inhibit the ability of agents such as lipopolysaccharides to stimulate an inflammatory response in murine macrophages (Arima et al., Biochemical Pharmacology 70:1506-1517 (2005); Motoyama et al., FEBS Letters 579:1707-1714 (2005)) and there appears to be interest in using these compounds to block sepsis in humans.

Since there are various forms of cyclodextrins with different side groups that appear to have different effects in mammalian macrophages, the effects of different cyclodextrins on gene expression in the trout macrophage cell cultures were tested. Trout macrophages were stimulated for 30 minutes to 24 hours with heptakis (2,6-di-O-methyl)-β-cyclodextrin (Heptakis); methyl-β-cyclodextrin (Methyl β); beta cyclodextrin (β CyD); 2-hydroxypropyl-β-cyclodextrin (Hydroxy β; 2-hydroxypropyl-α-cyclodextrin (Hydroxy α); gamma cyclodextrin (γ CyD). RNA was extracted and RT-PCR conducted with IL-1B and IL-6.

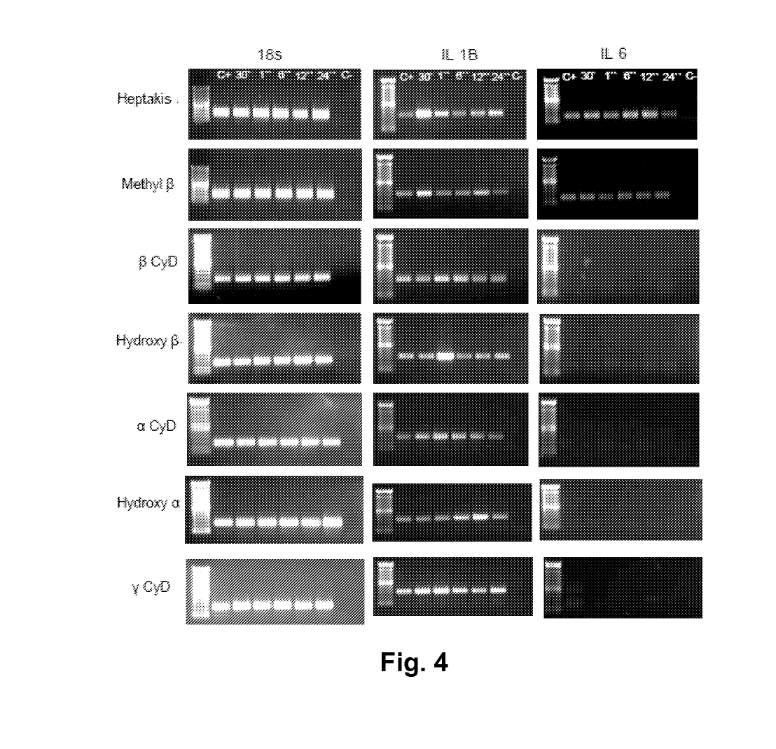

As shown in FIG. 4, 18s was a RNA loading control and the molecular weight standard is in the first lane. The most effective form of cyclodextrin was the gamma form on IL-1B expression though the heptakis form also stimulated IL-1B and 1L-6 that may indicate that different forms are stimulating the cell in different ways (FIG. 4). From these cell experiments, it was shown that cyclodextrin could up-regulate the innate immune system and could possibly be used as an immune agent to protect fish against pathogen challenges.

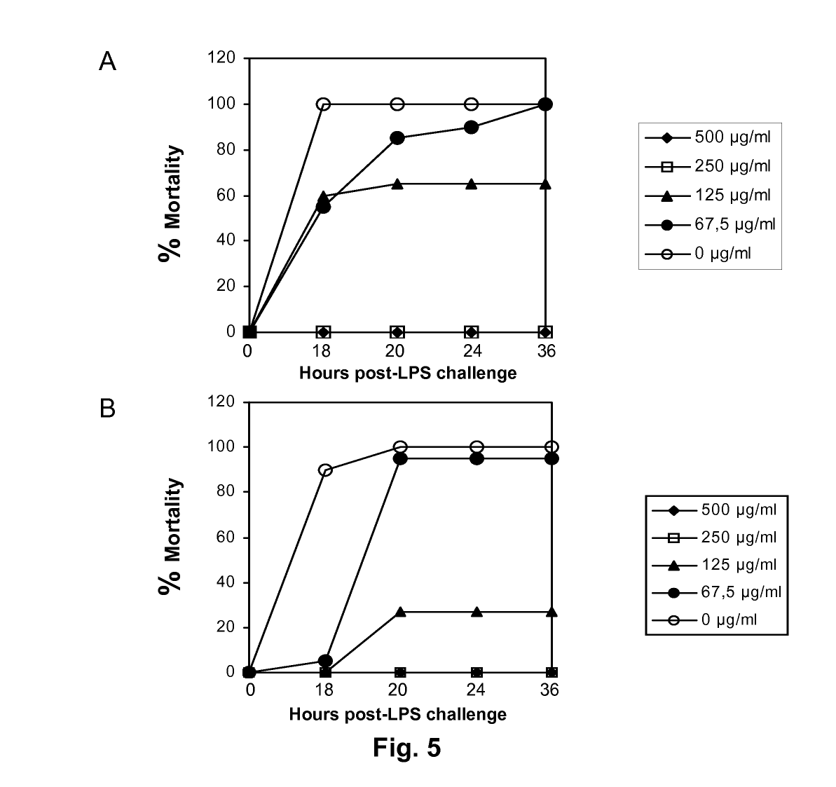

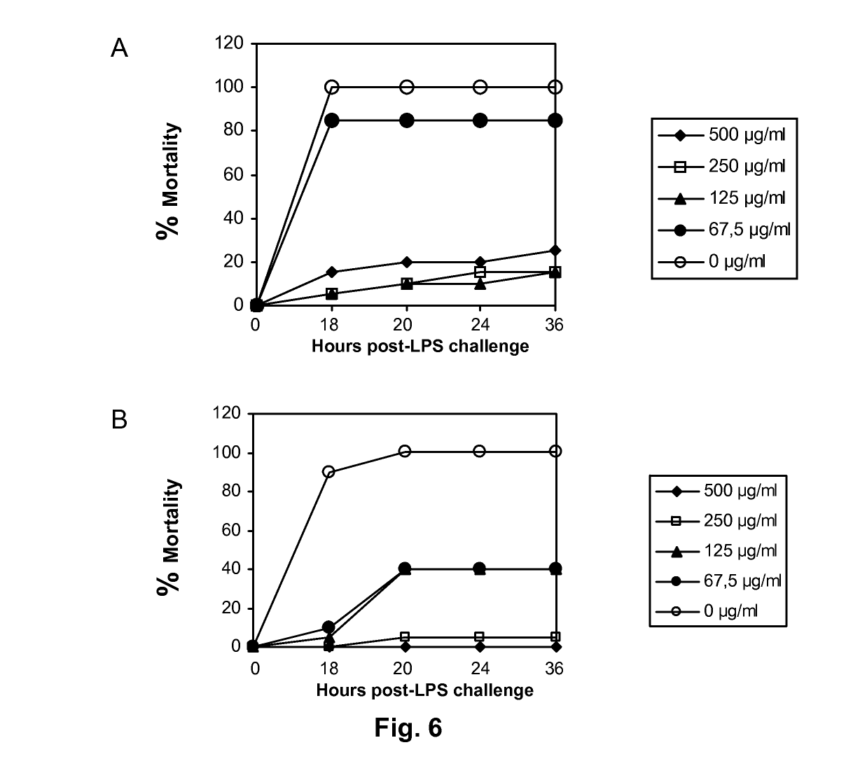

To test this, zebrafish larvae, 2 days post fertilization (dpf), were bathed with 0, 67.5, 125, 250 or 500 μg/mL of beta or alpha cyclodextrin. Zebrafish larvae were then bathed with a lethal concentration (i.e., 100% mortality) of bacterial lipopolysaccharide (LPS) (150 μg/mL Pseudomonas aeurginosa). LPS is part of the bacterial wall of Gram negative bacteria and thus acts like the bacteria though it is not alive. LPS concentrations of 150 μg/mL of Pseudomonas aeurginosa were reproducibly lethal concentrations for wild type zebrafish embryos (see FIGS. 5A-5B and 6A-6B). Both alpha and beta cyclodextrins protected the larvae against the LPS and this was complete at certain concentrations, in particular at concentrations of 250-500 μg/mL (FIGS. 5 and 6).

### Example 3

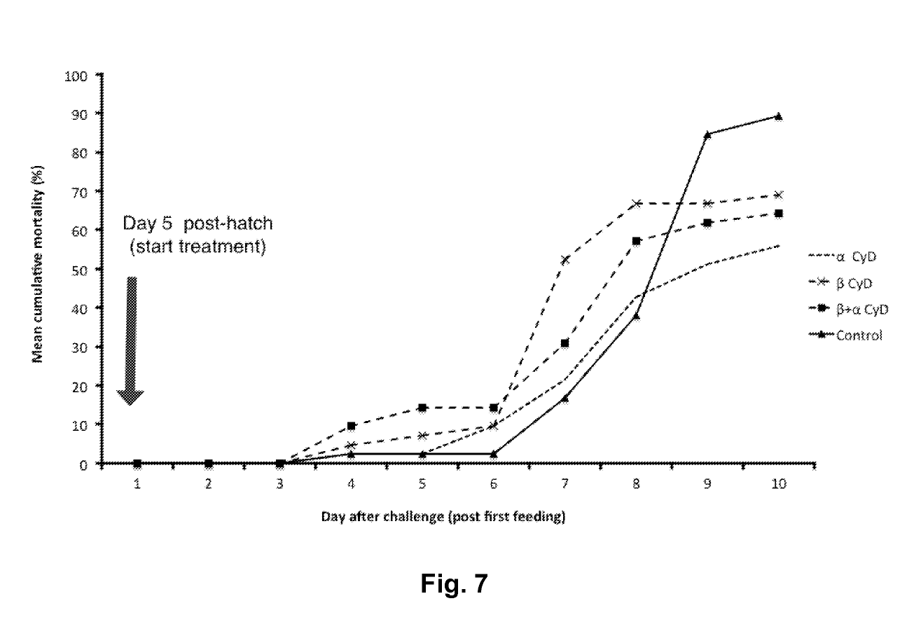

The larval viability during cyclodextrin treatment was explored. The survival during treatment was compared with normal larval growth condition after first feeding (day 5 post hatch). 6 independent batches by reproduction (84 embryos by batch) of zebrafish larvae were bathed with 0 (“Control”; normal larval growth condition) or 500 μg/mL of beta cyclodextrin (“β CyD”), alpha cyclodextrin (“α CyD”) or both (“β+α CyD”; 50/50% of each). Larval survival was recorded 10 days post first feeding (5 days post hatching) and was increased with all cyclodextrin treatments (FIG. 7). FIG. 7 shows survival toxicity data of cyclodextrin treatment post from first feeding. Larvae motility and larvae morphology were also measured and showed no discernible difference from control larvae groups.

It appears that cyclodextrins can provide protection against LPS in fish. The data on trout macrophages indicate that these compounds can stimulate gene expression so one possibility is that this up-regulation of immune regulators is responsible for the protection. However, it is also possible that the up-regulation of the cytokines seen in the macrophages following cyclodextrin stimulation, subsequently up-regulate other systems such as receptors, intracellular pathways or other immune components (e.g., complement) that would be responsible for interacting with and killing pathogens; heightening any subsequent challenge.

### Prophetic Example 4

### Cyclodextrin Diet Trials

The ability of cyclodextrin in diet to increase immune defence against viral and bacterial infection is evaluated in Atlantic salmon Salmo salar, life stage freshwater parr.

Phase 1 Study

Juvenile salmon, Salmo salar, of approximately 50 g are obtained and held at the Institute of Aquaculture, University of Stirling, UK. The fish are randomly distributed in fibreglass tanks (10 fish per tank) that is duplicated/treatment (n=8) using re-circulating fresh water circuits under a photoperiod of 12 hr light/12 hr dark and natural conditions of temperature. Fish are acclimatized to laboratory conditions for 15 days before being used for experiments.

Pellet diets are top dressed with 3 different concentrations of β-cyclodextrin (range is expected at 5-50-500 mg/Kg) in group sizes of 10 minimising the 3R's (reduction, refinement and replacement) of animal research and testing (n=10/group). The study is run over 2 weeks with the highest concentration diet being run over 4 weeks. Feeding follows commercial ration sizes.

Palatability/appetite studies are carried out by recovering excess feed from tanks and calculating feed intake. Fish behaviour is continuously monitored and recorded throughout the experimental period. Analysis includes swimming activity, tank distribution and response to stress stimuli e.g. escape response to netting. A pathological exam is carried out at the finalisation of the trial to identify lesions, intestinal rigidity, integrity and status of internal organs.

At the end of the experimental period fish are sacrificed following approved ethical protocols (lethal concentration of MS-222, 100 ppm, stage III of anaesthesia), and tissues (skin, gills, intestine and spleen) collected. Tissues removed for RNA extraction are frozen in liquid nitrogen and stored at −80° C. RNA isolation and RT-quantitative PCR are performed as described above.

### Prophetic Example 5

### Use of Cyclodextrin in Vaccines for Vibrio anguillarum

Vibrio anguillarum (also known as Listonella anguillarum) is a gram-negative bacterium that is the causative agent of vibriosis, a deadly disease affecting various marine and fresh-water fish, bivalves and crustaceans. Vibriosis is a hemorrhagic septicemia that is fatal to many aquatic species of aquaculture importance. A number of vaccines have been developed to this pathogen that have varying efficacy depending on the route of administration. It is possible that cyclodextrin(s) could increase the efficacy of these vaccines.

Rainbow trout juveniles are vaccinated by IP injection, water immersion, and through the diet, with a commercially available Vibrio vaccine, in the presence and absence of cyclodextrin(s) added to the vaccine as an adjuvant. Vaccine doses will follow manufacturers' instructions but different cyclodextrins including heptakis(2,6-di-O-methyl)-β-cyclodextrin; methyl-β-cyclodextrin; beta cyclodextrin; alpha cyclodextrin; 2-hydroxypropyl-β-cyclodextrin; 2-hydroxypropyl-α-cyclodextrin; and gamma cyclodextrin are tested with the vaccine at several concentrations.

Vaccinated and nonvaccinated trout are bath-challenged for 60 minutes with a virulent Vibrio serotype passaged and isolated from rainbow trout. Preliminary trials are conducted to determine the appropriate levels of Vibrio to use for bath challenges. Following challenge, the fish are returned to tanks and mortality recorded in each treatment over a 30 day period. Comparisons are made between vaccinated and non-vaccinated fish and between vaccination in the presence or absence of cyclodextrin(s).

### Prophetic Example 6

### Application of Cyclodextrin(s) in the Water for Protection Against Disease in Tropical Fish

Zebrafish larvae and adults are held in aquaria containing water with various types of cyclodextrins disclosed herein at concentrations from 0-1,000 μg/ml. Experiments will be run with continual exposure to cyclodextrin(s) and with exposures for short durations (days to weeks) prior to pathogen challenge.

Zebrafish are challenged with a bacterial pathogen such as Flexibacter columnaris (cotton wool disease) or with common parasites such as Ichthyophthirius multifiliis (cause of ick). The protection afforded by the cyclodextrin is assessed by monitoring the acquisition of disease and mortality. The relationship of disease protection with type of cyclodextrin, dose and duration of treatment is determined. Cyclodextrins are expected to provide increased protection against disease and mortality.

### Prophetic Example 7

### Application of Cyclodextrin(s) in the Water for Treating Diseases in Tropical Fish

Zebrafish are challenged with a bacterial pathogen such as Flexibacter columnaris, (cotton wool disease) or with common parasites such as Ichthyophthirius multifiliis (cause of ick).

After symptoms of disease are evident, fish are dosed continuously in the water with various cyclodextrins disclosed herein at concentrations from 0-1,000 μg/ml. Cyclodextrins are expected to ameliorate disease in fish.

It is to be understood that the invention is not limited in its application to the details of construction and the arrangement of components set forth in the description or illustrated in the drawings. The invention is capable of other embodiments and of being practiced or of being carried out in various ways.

Various features and advantages of the invention are set forth in the following claims.

1. A method of stimulating the innate immune system of fish, the method comprising feeding the fish a composition comprising an amount of cyclodextrin effective to stimulate the innate immune system of the fish with the proviso that the composition does not comprise cysteamine.

2. A method of stimulating the innate immune system of fish, the method comprising combining the fish with water comprising cyclodextrin at a concentration effective to stimulate the innate immune system of the fish.

3. The method of claim 1, wherein cyclodextrin induces IL-1β expression.

4. A method to enhance the efficacy of a fish vaccine, the method comprising administering a cyclodextrin to a fish treated with the vaccine.

5. The method of claim 1, wherein the incidence of disease is reduced in the fish.

6. The method of claim 1, wherein the fish is continuously administered the cyclodextrin.

7. The method of claim 1, wherein the cyclodextrin is administered to the fish before a stress event.

8. The method of claim 1 wherein the effective amount of cyclodextrin is between about 5 mg/kg to about 1000 mg/kg.

9. The method of claim 1, wherein the fish is tropical fish.

10. The method of claim 1, wherein the fish is zebrafish, salmon or rainbow trout.

11. The method of claim 1, wherein the disease is caused by a bacterium or a virus.

12. The method of claim 1, wherein the disease is caused by a gram-negative bacterium.

13. The method of claim 1, wherein the disease is cause by Pseudomonas aeruginosa.

14. The method of claim 1, wherein the cyclodextrin comprises α-cyclodextrin, β-cyclodextrin, or γ-cyclodextrin.

15. The method of claim 1, wherein the cyclodextrin comprises β-cyclodextrin.

16. A composition for feeding fish, the composition comprising cyclodextrin and a component that is digestible by fish or non-toxic to fish, with the proviso that the composition does not comprise cysteamine.

17. The composition of claim 16, wherein the cyclodextrin comprises β-cyclodextrin.

18.-19. (canceled)
